(19) United States
(12) Patent Application Publication
(10) Pub. No.: US 2022/0125832 A1
(43) Pub. Date: Apr. 28, 2022

(54) USE OF PRUSSIAN BLUE NANOPARTICLES IN THE PREPARATION OF A MEDICAMENT FOR THE PREVENTION, DELAY OR TREATMENT OF NEURODEGENERATIVE DISEASE

(71) Applicant: Tianjin University, Nankai District, Tianjin (CN)

(72) Inventors: Jin Chang, Nankai District (CN); Yan Dou, Heping District (CN); Dongju Zhao, Nankai District (CN); Yuqing Tang, Nankai District (CN)

(73) Assignee: Tianjin University, Nankai District, Tianjin (CN)

(21) Appl. No.: 17/257,076

(22) PCT Filed: Sep. 18, 2020
(86) PCT No.: PCT/CN2020/116031

§ 371 Date: Dec. 30, 2020

(51) Int. Cl. A61K 33/26; A61K 47/64; A61K 47/68; A61K 47/18; A61K 9/14; A61P 25/28

(52) U.S. Cl. CPC A61K 33/26; A61K 47/644; A61K 47/64; A61P 25/28; A61K 47/18; A61K 9/14; A61K 47/6843

(57) ABSTRACT

The present application relates to use of Prussian blue nanoparticles in the preparation of a medicament for the prevention, delay or treatment of neurodegenerative disease. The present application finds that Prussian blue nanoparticles have significant effects in the prevention, delay or treatment of neurodegenerative disease. Cell test results show that Prussian blue nanoparticles can reduce the level of ROS in nerve cells stimulated by hydrogen peroxide and increase the proportion of living cells in nerve cells stimulated by hydrogen peroxide. Animal test results show that Prussian blue nanoparticles can significantly reduce the expression level of oxidative stress markers in the hippocampus of mouse models of neurodegenerative disease, and significantly improve the learning and memory abilities and ameliorate motor dysfunction of mouse models of neurodegenerative disease. Prussian blue nanoparticles have advantages of simple preparation process, easy for large scale production, mild reaction conditions and easy for surface modification.

### TECHNICAL FIELD

The present application belongs to the field of biomedical technology, relates to new use of Prussian blue nanoparticles, and specifically, relates to use of Prussian blue nanoparticles in the preparation of a medicament for the prevention, delay or treatment of neurodegenerative disease.

### BACKGROUND

Neurodegenerative disease is an irreversible nerve system disease caused by the loss of neuronal cells in the brain and spinal cord, including Alzheimer's disease, Parkinson's disease, Amyotrophic lateral sclerosis, Huntington's disease, etc. With the advent of aging society, the incidence of neurodegenerative disease is rising, causing huge social medical expenditure and family pressure. The effective treatments for most neurodegenerative diseases are still unavailable. Therefore, finding effective methods to prevent, delay and treat such diseases is a problem that needs to be resolved urgently.

Oxidative stress plays a key role in the occurrence and development of neurodegenerative disease. Oxidative stress refers to that oxidation and anti-oxidation in the body are unbalanced and a large amount of reactive oxygen species (ROS) and reactive nitrogen species are accumulated, causing molecular oxidation and tissue damage, and ultimately leading to diseases. Compared with other tissues, the brain tissue is more susceptible to ROS, and the factor of aging further weakens the function of the antioxidant system in the brain such as superoxide dismutase, catalase and peroxidase such that ROS is significantly increased and cannot be effectively removed, aggravating oxidative stress. Studies have shown that excessive ROS will disrupt the intracellular calcium ion balance, and damage synapsis by regulating the release of neurotransmitters from the presynaptic terminal and postsynaptic neuronal responses, affecting the neuronal signal transduction in the brain. Meanwhile, oxidative stress can mediate apoptosis of neuronal cells by regulating the expression of apoptosis-related proteins such as Bcl-2. In addition, oxidative stress will activate glial cells and further induce the release of proinflammatory factors, thereby causing the damage and loss of neurons through neurogenic inflammation. Therefore, oxidative stress plays an important role in the occurrence and development of neurodegenerative disease such as Alzheimer's disease, Parkinson's disease, Amyotrophic lateral sclerosis, Huntington's disease, etc.

Prussian blue nanoparticles have advantages of simple preparation process, mild reaction conditions and easy for surface modification, and because of their rich redox potentials and the unique characteristic of electron spin, their use in the field of biomedicine has become a research hotspot in recent years. For example, CN105288665A discloses a Prussian blue nanoparticle contrast agent. The contrast agent comprises a Prussian blue nanoparticle core and a polyethylene glycol shell layer coated on the surface of the Prussian blue nanoparticle. The water solubility and biocompatibility of the Prussian blue nanoparticle contrast agent are good, which is conducive to its application in organisms. For example, CN105477648A discloses a Prussian blue-like nanoparticle that targets lymph and a preparation method thereof. According to the method, diethylenetriamine pentaacetic acid is cross-linked with hyaluronic acid and chelated on gadolinium ions to form a stable lymph-targeting Prussian blue-like nanoparticle with hyaluronic acid on the surface. The core, Prussian blue-like nanoparticle, has more unpaired electrons by using gadolinium to substitute the position of ferric iron, and generates stronger magnetic resonance signal. The surface of the nanoparticle is coated with hyaluronic acid, and since the hyaluronic acid is one of human tissue composition, the biocompatibility of the nanoparticle is very good.

However, there is no relevant report on the use of Prussian blue nanoparticles in the preparation of a medicament for the prevention, delay or treatment of a neurodegenerative disease.

### SUMMARY

The present application provides new use of Prussian blue nanoparticles, and specifically provides use of Prussian blue nanoparticles in the preparation of a medicament for the prevention, delay or treatment of neurodegenerative disease.

According to the first aspect, the present application provides use of Prussian blue nanoparticles in the preparation of a medicament for the prevention, delay or treatment of neurodegenerative disease.

The new use of Prussian blue nanoparticles involved in the present application includes three aspects: the first one is the use of Prussian blue nanoparticles in the preparation of a medicament for the prevention of neurodegenerative disease; the second one is the use of Prussian blue nanoparticles in preparation of a medicament for the delay of neurodegenerative disease; and the third one is the use of Prussian blue nanoparticles in the preparation of a medicament for the treatment of neurodegenerative disease. The cell test results of the present application show that Prussian blue nanoparticles can reduce the level of ROS in nerve cells stimulated by hydrogen peroxide, and increase the proportion of living cells in the nerve cells stimulated by hydrogen peroxide; the animal test results show that Prussian blue nanoparticles can significantly reduce the expression level of oxidative stress markers in the hippocampus of mouse models of neurodegenerative disease, significantly reduce the expression level of inflammatory-related molecules in the hippocampus of the mouse models of neurodegenerative disease, and significantly improve the learning and memory abilities of the mouse models of neurodegenerative disease.

The Prussian blue nanoparticles involved in the present application can be prepared by those skilled in the art according to conventional methods disclosed in the existing art. The present application does not specifically limit the preparation method of Prussian blue nanoparticles. Exemplarily, Prussian blue nanoparticles can be prepared by a one-step method, and the specific preparation steps are as follows:

(1) potassium ferrocyanide and carboxylated polyethylene glycol are separately dissolved in deionized water and mixed thoroughly to obtain a clear solution A; ferric chloride is thoroughly dissolved in deionized water to obtain a clear solution B; and the solution B is added dropwise to the solution A such that the molar ratio of potassium ferrocyanide to ferric chloride is 1:1, and the resulting mixed solution is reacted at 40-80° C. for 0.5-2 h; and

(2) the reaction system is cooled to 20-30° C., then the reaction system is reacted for 0.5-2 h, centrifuged and washed to obtain non-functionally modified Prussian blue nanoparticles.

In some embodiments of the present application, the neurodegenerative disease includes Alzheimer's disease, Parkinson's disease, Amyotrophic lateral sclerosis, Huntington's disease, spinocerebellar ataxia, spinal muscular atrophy, multiple sclerosis or epilepsy.

The pathogenesis of the above-mentioned neurodegenerative disease is related to oxidative stress, that is, the oxidation and anti-oxidation in the body become unbalanced and a large amount of reactive oxygen species (ROS) and reactive nitrogen species are accumulated, causing molecular oxidation and tissue damage; excessive ROS disrupts the intracellular calcium ion balance, and damages synapsis by regulating the release of neurotransmitters from the presynaptic terminal and postsynaptic neuronal responses, affecting the neuronal signal transduction in the brain; and meanwhile, glial cells are activated, inducing the release of proinflammatory factors, thereby causing the damage and loss of neurons, and ultimately leading to the occurrence of the above-mentioned disease.

In some embodiments of the present application, the Prussian blue nanoparticles are Prussian blue nanoparticles which are functionally modified or which are non-functionally modified.

Prussian blue nanoparticles have advantages of simple preparation process, mild reaction conditions and easy for surface modification. Those skilled in the art can perform functional modification on the surface of nanoparticles according to actual application requirements.

In some embodiments of the present application, the Prussian blue nanoparticles are Prussian blue nanoparticles which are modified with a functional molecule which crosses the blood-brain barrier and/or a molecule which specifically targets amyloid-β (Aβ) deposition.

In some embodiments of the present application, the functional molecule which crosses the blood-brain barrier includes any one or any combination of at least two of transferrin, lactoferrin, apolipoprotein E (Apo E), Angiopep-2, RVG29 or a TAT peptide. The combination of the at least two of the above, for example, may be a combination of transferrin and lactoferrin, a combination of Angiopep-2 and RVG29, a combination of RVG29 and TAT peptide, etc. Any other combinations can also be selected, which will not be further described herein.

In some embodiments of the present application, the molecule which specifically targets Aβ deposition includes any one or any combination of at least two of Congo red, thioflavin S or an anti-Aβ antibody. The combination of the at least two of the above, for example, may be a combination of Congo red and thioflavin S, a combination of thioflavin S and anti-Aβ antibody, etc. Any other combinations can also be selected, which will not be further described herein.

In some embodiments of the present application, the Prussian blue nanoparticles have a particle size of 80-200 nm, for example, 80 nm, 100 nm, 120 nm, 140 nm, 150 nm, 160 nm, 180 nm or 200 nm. Any other specific values within the above range can also be selected, which will not be further described herein.

In some embodiments of the present application, the Prussian blue nanoparticles are loaded on a pharmaceutical carrier.

The pharmaceutical carrier is, for example, a liposome, a micelle, a dendrimer, a microsphere or a microcapsule.

In some embodiments of the present application, the Prussian blue nanoparticles are included in a pharmaceutical composition.

The Prussian blue nanoparticles involved in the present application may also be combined with an additional biologically active ingredient capable of preventing, delaying or treating neurodegenerative disease in different proportions to form a pharmaceutical composition, in which the additional biologically active ingredient and the Prussian blue nanoparticles can cooperates with each other to exert the effect.

In some embodiments of the present application, the medicament is in a dosage form comprising a tablet, a powder, a suspension, a granule, a capsule, an injection, a spray, a solution, an enema, an emulsion, a film, a suppository, a patch, a nasal drop or a pill.

The Prussian blue nanoparticles described in the present application can be administered alone or in combination with an adjuvant to form an appropriate dosage form for administration. The adjuvant includes any one or any combination of at least two of a diluent, an excipient, a filler, a binder, a wetting agent, a disintegrant, an emulsifier, a cosolvent, a solubilizer, an osmotic pressure regulator, a surfactant, a pH regulator, an antioxidant, a bacteriostatic agent, or a buffer. The combination of at least two of the above, for example, is a combination of diluent and excipient, a combination of emulsifier and cosolvent, a combination of filler and wetting agent, etc.

When the dosage form is a tablet, an excipient can be included, such as microcrystalline cellulose, starch, or calcium carbonate, etc.; and a disintegrant can also be included, such as croscarmellose sodium, etc. When the dosage form is a capsule, a hard capsule or a soft capsule can be prepared, and the Prussian blue nanoparticles and adjuvants can be prepared in a form of powders or granules and filled into the capsule. When the dosage form is a suspension, flavoring agents and suspending agents can be added to adjust the taste and mouthfeel. When the dosage form is an emulsion, emulsifiers and cosolvents can be appropriately added to adjust the solubility and emulsifiablility for administration.

In some embodiments of the present application, the medicament is administrated by a route comprising intravenous injection, intraperitoneal injection, intramuscular injection, subcutaneous injection, oral administration, sublingual administration, nasal administration or transdermal administration.

Oral administration is generally carried out in the form of tablets or capsules. In addition, when the medicament is orally administered in the form of tablets or capsules, the tablets or capsules can be prepared as controlled-release preparations or sustained-release preparations. According to the required medicinal effect and action time, the appropriate dose of controlled release adjuvants or sustained release adjuvants is selected.

According to the second aspect, the present application provides use of Prussian blue nanoparticles in the preparation of an expression inhibitor of an oxidative stress marker, wherein the oxidative stress marker includes 4-hydroxynonenal, malondialdehyde or 8-hydroxyguanosine.

The present application further provides use of Prussian blue nanoparticles in the preparation of an expression inhibitor of an astrocyte marker GFAP, microglial cell marker Iba-1, inflammatory factor TNF-α, inflammatory factor IL-1β, apoptosis protein P53 or Caspase-3.

The present application further provides use of Prussian blue nanoparticles in the preparation of an expression promoter of a synaptic damage marker SYN1, synaptic damage marker PSD95 or apoptosis protein Bcl-2.

According to the third aspect, the present application provides a medicament for the prevention, delay or treatment of neurodegenerative disease. The medicament for the prevention, delay or treatment of neurodegenerative disease includes Prussian blue nanoparticles.

According to the fourth aspect, the present application provides use of Prussian blue nanoparticles in the prevention, delay or treatment of neurodegenerative disease.

Compared with the existing art, the present application has beneficial effects below.

The Prussian blue nanoparticles involved in the present application have significant effects in the preparation of a medicament for the prevention, delay or treatment of neurodegenerative disease. The cell test results of the present application show that Prussian blue nanoparticles can reduce the level of ROS in nerve cells stimulated by hydrogen peroxide, and increase the proportion of living cells in the nerve cells stimulated by hydrogen peroxide; the animal test results show that Prussian blue nanoparticles can significantly reduce the expression level of oxidative stress markers in the hippocampus of mouse models of neurodegenerative disease, significantly reduce the expression level of inflammatory-related molecules in the hippocampus of the mouse models of neurodegenerative disease, and significantly improve the learning and memory abilities of the mouse models of neurodegenerative disease. Prussian blue nanoparticles has advantages of simple preparation process, easy for large scale production, mild reaction conditions and easy for surface modification.

### BRIEF DESCRIPTION OF DRAWINGS

FIG. 1 is a transmission electron microscopic image of double-targeting Prussian blue nanoparticles;

FIG. 2 is a particle size characterization diagram of double-targeting Prussian blue nanoparticles;

FIG. 3 is a potential characterization diagram of double-targeting Prussian blue nanoparticles;

FIG. 4 is a diagram showing results of ROS levels in each group of cells in reactive oxygen species detection;

FIG. 5 is a diagram showing results of apoptosis levels, detected by using the Annexin V-FITC/PI kit;

FIG. 6 is a diagram showing results of expression levels of pathological characteristic markers, detected by western blotting; and

FIG. 7 is a diagram showing results of the water maze test.

### DETAILED DESCRIPTION

The technical solutions of the present application are further described below through specific examples. Those skilled in the art should understand that the examples described herein are merely used for a better understanding of the present application and should not be construed as specific limitations to the present application.

All the raw materials and reagents used in the following examples are commercially available or can be prepared according to common general knowledge of those skilled in the art.

Nerve cell strain PC12 was donated by the Tianjin Medical University General Hospital, and APP/PS1 transgenic mice and C57BL/6 mice were purchased from Beijing HFK Bioscience Co., Ltd.

### Example 1

In this example, non-functionally modified Prussian blue nanoparticles were prepared. The preparation method includes steps described below.

(1) 0.01 mM of potassium ferrocyanide and 0.001 mM of mPEG-COOH (MW5000) were separately dissolved in 5 mL of deionized water and mixed thoroughly to obtain a clear solution A; 0.01 mM of ferric chloride was thoroughly dissolved in 5 mL of deionized water to obtain a clear solution B; and the solution B was added dropwise to the solution A and reacted at 60° C. for 1 h.

(2) The reaction system was cooled to 25° C., then the reaction system was reacted for 1 h, centrifuged and washed to obtain non-functionally modified Prussian blue nanoparticles.

The prepared Prussian blue nanoparticles were characterized with respect to particle size and potential, and results are as follows: the particle size measured by dynamic light scattering was 80 nm, and the surface potential was −32 mV.

### Example 2

In this example, single-targeting modified Prussian blue nanoparticles were prepared. The preparation method includes steps described below.

(1) 0.01 mM of potassium ferrocyanide and 0.001 mM of mPEG-COOH (MW5000) were separately dissolved in 5 mL of deionized water and mixed thoroughly to obtain a clear solution A; 0.01 mM of ferric chloride was thoroughly dissolved in 5 mL of deionized water to obtain a clear solution B; and the solution B was added dropwise to the solution A and reacted at 60° C. for 1 h.

(2) The reaction system was cooled to 25° C., then the reaction system was reacted for 1 h, centrifuged and washed to obtain non-functionally modified Prussian blue nanoparticles.

(3) The obtained Prussian blue nanoparticles and 0.01 mM of 1-(3-dimethylaminopropyl)-3-ethylcarbodiimide hydrochloride were dissolved in 10 mL of deionized water, and reacted at 25° C. for 15 min to obtain a solution D.

(4) 0.01 mM of N-hydroxysuccinimide and 0.4 μM of Congo red were added to the solution D, reacted at 25° C. for 24 h, then centrifuged and washed, and the resulting precipitate was resuspended in deionized water to obtain single-targeting Prussian blue nanoparticles which specifically targeted AP deposition.

The prepared Prussian blue nanoparticles were characterized with respect to particle size and potential, and results are as follows: the particle size measured by dynamic light scattering was 130 nm, and the surface potential was −24 mV.

### Example 3

In this example, double-targeting modified Prussian blue nanoparticles were prepared. The preparation method includes steps described below.

(1) 0.01 mM of potassium ferrocyanide and 0.001 mM of mPEG-COOH (MW5000) were separately dissolved in 5 mL of deionized water and mixed thoroughly to obtain a clear solution A; 0.01 mM of ferric chloride was thoroughly dissolved in 5 mL of deionized water to obtain a clear solution B; and the solution B was added dropwise to the solution A and reacted at 60° C. for 1 h.

(2) The reaction system was cooled to 25° C., then the reaction system was reacted for 1 h, centrifuged and washed to obtain non-functionally modified Prussian blue nanoparticles.

(3) The obtained Prussian blue nanoparticles and 0.01 mM of 1-(3-dimethylaminopropyl)-3-ethylcarbodiimide hydrochloride were dissolved in 10 mL of deionized water and reacted at 25° C. for 10 min to obtain a solution D.

(4) 0.01 mM of N-hydroxysuccinimide, 0.005 mg of transferrin and 0.5 μmM of Congo red were added to the solution D, reacted at 25° C. for 24 h, then centrifuged and washed, and the resulting precipitate was resuspended in deionized water to obtain double-targeting Prussian blue nanoparticles which can cross the blood-brain barrier and specifically target AP deposition.

The obtained double-targeting Prussian blue nanoparticles were characterized through the following characterization tests.

(I) Transmission electron microscopy characterization: results are shown in FIG. 1, and it can be seen from the figure that the nanoparticles were spherical and had uniform particle size and good dispersion.

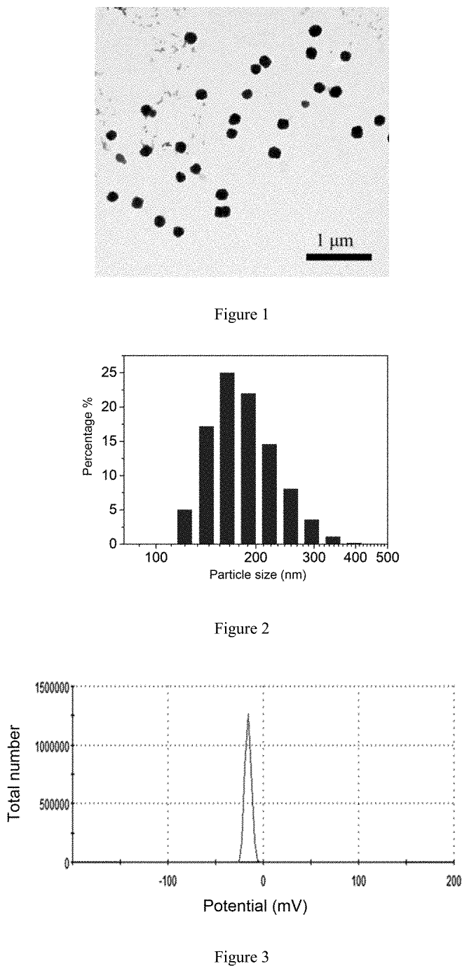

(II) Particle size and potential characterization: results are shown in FIGS. 2 and 3, respectively, and it can be seen from the figures that the particle size of the nanoparticles was 188 nm, and the potential was −15.8 mV

### Example 4

In this example, the effect of the Prussian blue nanoparticles prepared in Example 3 on the prevention, delay or treatment of Alzheimer's disease was evaluated.

(I) Cell Test

Nerve cell strain PC12 was used as an experimental subject to construct an Alzheimer's disease prevention model and an Alzheimer's disease treatment model respectively. The method of constructing the Alzheimer's disease prevention model is as follows: PC12 cells were seeded in a 24-well plate at a density of 4×10⁵ cells per well; after the cells had grown to about 80%, a medium containing 500 μL of 10 μg/mL double-targeting Prussian blue nanoparticles was added and incubated at 37° C. for 24 h, and then the medium was removed; and 500 μL of medium containing 200 μM of hydrogen peroxide was added and incubated at 37° C. for 24 h. The method of constructing the Alzheimer's disease treatment model is as follows: PC12 cells were seeded in a 24-well plate at a density of 4×10⁵ cells per well; after the cells had grown to about 80%, 500 μL of medium containing 200 μM of hydrogen peroxide was added and incubated at 37° C. for 24 h, and then the medium was removed; and 500 μL of medium containing 10 μg/mL double-targeting Prussian blue nanoparticles was added and incubated at 37° C. for 24 h. Cells without any treatment, cells incubated with a hydrogen peroxide solution alone, and cells incubated with a double-targeting Prussian blue nanoparticle solution alone were used as controls.

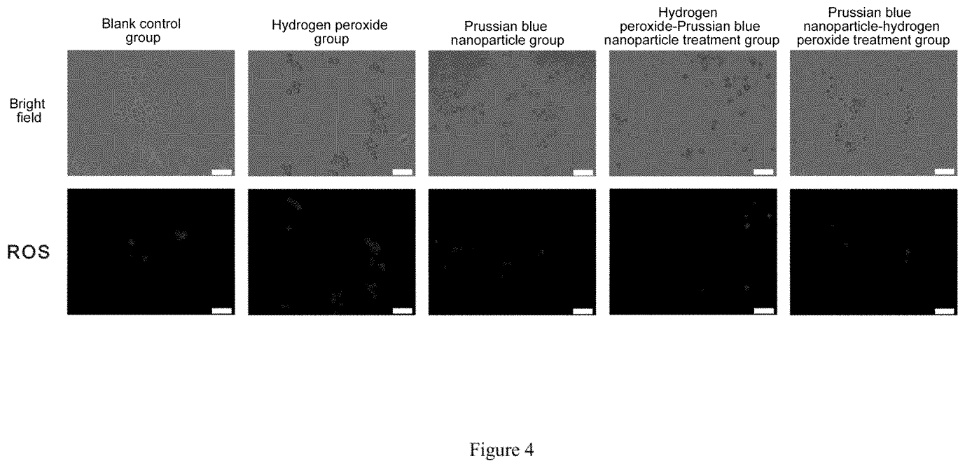

The intracellular ROS level was detected by using a Reactive oxygen species assay kit: The cells were washed 3 times with PBS, and a medium containing 10 μM of DCFH-DA was added to the culture dish and incubated for 20 min. The cells were observed under an inverted fluorescence microscope and the fluorescence pictures were recorded to study the anti-oxidative stress function of the nanoparticle at the cellular level. The results are shown in FIG. 4 (with the scale of 50 μm). It can be seen from the results in FIG. 4 that: compared with the blank control group, the hydrogen peroxide treatment group showed enhanced green fluorescence signal and increased intracellular ROS level, and the intracellular ROS level of the double-targeting Prussian blue nanoparticle treatment group did not significantly reduced; and for the group of cells which were first incubated with the hydrogen peroxide and then incubated with the double-targeting Prussian blue nanoparticles (hydrogen peroxide-double-targeting Prussian blue nanoparticle incubation group) and the group of cells which were first incubated with the double-targeting Prussian blue nanoparticles and then incubated with the hydrogen peroxide (double-targeting Prussian blue nanoparticle-hydrogen peroxide incubation group), the ROS levels of cells in both groups were lower than the ROS level of the hydrogen peroxide treatment group, indicating that the double-targeting Prussian blue nanoparticles can reduce the ROS level in nerve cells stimulated by hydrogen peroxide.

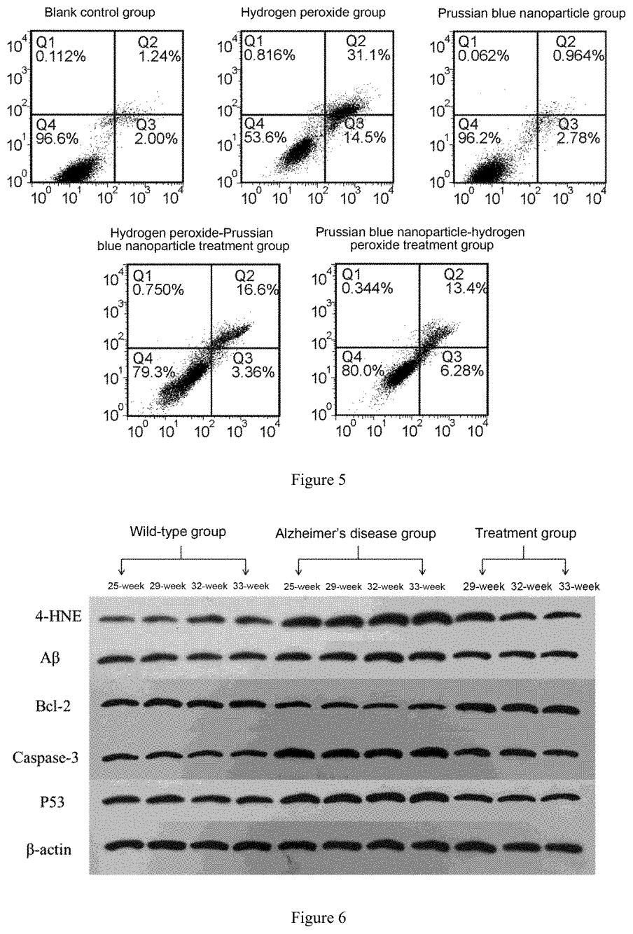

The apoptosis level was detected by using an Annexin V-FITC/PI apoptosis detection kit: the cells were collected and resuspended in PBS, 5 μL of Annexin V-FITC was added and incubated at 25° C. for 10 min in the dark, and then 5 μL of PI was added. The apoptosis analysis was carried out on the flow cytometer by using Flow Jo analysis software. The impact of the nanoparticles on cell apoptosis was studied, and the results are shown in FIG. 5. It can be seen from the results in FIG. 5 that, compared with the blank control group, the hydrogen peroxide treatment group would cause cell apoptosis, in which the proportion of living cells was 53.6%, while the proportion of living cells in the double-targeting Prussian blue nanoparticle treatment group basically was not impacted, indicating that double-targeting Prussian blue nanoparticles have good safety. For the hydrogen peroxide-double-targeting Prussian blue nanoparticle incubation group and the double-targeting Prussian blue nanoparticle-hydrogen peroxide incubation group, the proportion of living cells in both groups of cells was about 80%, lower than the proportion of living cells of the hydrogen peroxide treatment group, indicating that the double-targeting Prussian blue nanoparticles can improve the proportion of living cells in nerve cells stimulated by hydrogen peroxide, and have a protective effect on nerve cells.

(II) Animal Test

APP/PS1 transgenic mice were used as Alzheimer's disease animal models to construct an Alzheimer's disease delay model and an Alzheimer's disease treatment model respectively.

The method of constructing the Alzheimer's disease treatment model is as follows. 25-week-old (female) APP/PS1 transgenic mice were used as Alzheimer's disease animal models for treatment test, and 25-week-old female C57BL/6 mice were used as control. The mice were divided into the following groups: (1) wild-type group: C57BL/6 mice; (2) Alzheimer's disease group: APP/PS1 mice; and (3) treatment group: APP/PS1 mice treated with double-targeting Prussian blue nanoparticles; and each group had 15 mice. For the treatment group, 50 μg of double-targeting Prussian blue nanoparticles were administered to mice by tail vein injection, once a week, for a total of 7 times. In the process of treatment, mice in each group were detected for relevant indexes before (25 weeks of age), during (29 weeks of age), and after (32 weeks of age, 33 weeks of age) treatment, respectively.

The total protein was extracted from the hippocampus of each group of mice. The expression level of pathological characteristic markers was detected by western blotting, including oxidative stress marker: 4-hydroxynonenal (4-HNE); and apoptosis-related proteins: P53, Caspase-3, Bcl-2; and the Aβ expression was also detected. The results are shown in FIG. 6. It can be seen from the figure that the expression of 4-hydroxynonenal, Aβ, P53 and Caspase-3 in the hippocampus of the mice in the treatment group decreased, and the expression of anti-apoptotic protein Bcl-2 increased, indicating that double-targeting Prussian blue nanoparticles can reduce the level of oxidative stress in the hippocampus of mice, reduce the expression of Aβ, and regulate the expression of apoptosis-related proteins, and have a protective effect on nerve cells in the brain.

After treatment, the learning and memory abilities of mice were evaluated by the water maze test, Y maze test and open field test. The specific methods are as follows. (1) Water maze test: the spatial learning and memory abilities of mice were detected by Morris water maze test, including place navigation test and space probe test. The place navigation test lasted for 5 days. The mice were placed into the water from 4 entry points once a day facing the pool wall, and the computer system recorded their swimming trajectories from the entry position to the end position within 60 s. In the space probe test, the platform was removed after the place navigation test, the mice were placed in the pool from any entry point, and the computer system recorded their swimming trajectories to examine the abilities of the mice for their memories about the original platform. (2) Y maze test: the Y maze consisted of 3 arms of the same length, which were called area I, area II, and area III respectively. The lower arm of Y maze (area I) was defined as the starting area, the left side (area II) was defined as the safe area, and the intersection of the three arms was defined as the isolation area (area 0). Before the test, the area III was closed, and areas I, II and 0 were open and allowed to freely enter. The tested mice were allowed to freely adapt to the maze for 5 min and then taken out, and let them rest for 30 min.

After that, the second test was carried out, in which all areas must be kept open and allowed to freely enter. The mice were put in the maze from the area I again and then taken out after 5 min. The number of times and duration of the mice entering each area were counted, and on the premise that the individual differences in the first test were small, data of the mice entering the area III was specifically analyzed statistically. (3) Open field test: with their heads facing the box wall, the mice were put into an open field analysis box. The surrounding environment was kept quiet. The movement of each mouse within 120 s was observed. The trajectory, moving distance and gait of each mouse were recorded.

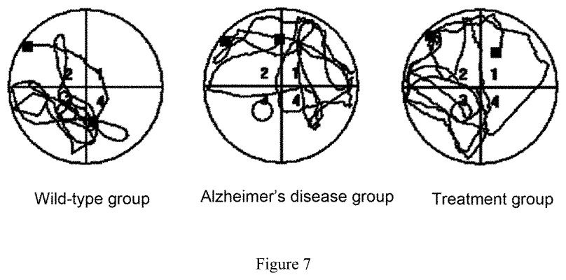

The results of the water maze test are shown in FIG. 7. It can be seen from the figure that the swimming trajectories of mice in the wild-type group and the treatment group showed the purpose of finding the platform, while the swimming trajectories of mice in the Alzheimer's disease group showed a phenomenon of circling, indicating that the treatment with double-targeting Prussian blue nanoparticles can improve the learning and memory ability of mice suffered from Alzheimer's disease.

The method of constructing the Alzheimer's disease delay model is as follows. 10-week-old (female) APP/PS1 transgenic mice were used as Alzheimer's disease delay animal models for test, and 10-week-old female C57BL/6 mice were used as control. The mice were divided into the following groups: (1) wild-type group: C57BL/6 mice; (2) Alzheimer's disease group: APP/PS1 mice; (3) delay group: APP/PS1 mice treated with double-targeting Prussian blue nanoparticles; and each group had 30 mice. For the delay group, 25 μg of double-targeting

Prussian blue nanoparticles were administered to mice by tail vein injection, once a week, for a total of 12 times. In the process of administration, mice in each group were detected for relevant indexes before (10 weeks of age), during (14 weeks of age, 18 weeks of age), and after (22 weeks of age, 23 weeks of age) administration, respectively.

The total protein was extracted from the hippocampus of each group of mice. The expression level of pathological characteristic markers was detected by western blotting, including oxidative stress markers: 4-hydroxynonenal, malondialdehyde, 8-hydroxyguanosine; apoptosis-related proteins: P53, Caspase-3, Bcl-2; inflammation-related: astrocyte marker GFAP, microglia marker Iba-1, inflammatory factors TNF-α and IL-1β; and synaptic damage markers: SYN1, PSD95; and the Aβ expression was also detected. The results showed that double-targeting Prussian blue nanoparticles can reduce the level of oxidative stress in mice suffered from Alzheimer's disease, reduce neuronal cell apoptosis, relieve inflammation, improve synaptic damage, and reduce the expression of Aβ.

Then, the learning and memory abilities of mice were evaluated through the water maze test, Y maze test and open field test, and the specific methods were the same as above.

The results showed that the swimming trajectories of mice in the wild-type group and the delay group showed the purpose of finding the platform, while the swimming trajectories of mice in the Alzheimer's disease group showed a phenomenon of circling, indicating that the double-targeting Prussian blue nanoparticles can delay the development of Alzheimer's disease.

The applicant has stated that although the use of Prussian blue nanoparticles in the preparation of a medicament for the prevention, delay or treatment of neurodegenerative disease in the present application is described through the above-mentioned examples, the present application is not limited to the above-mentioned examples, which means that the implementation of the present application does not necessarily depend on the above-mentioned examples. It should be apparent to those skilled in the art that any improvements made to the present application, equivalent replacements of raw materials of the product of the present application, additions of adjuvant ingredients to the product of the present application, and selections of specific manners, etc., all fall within the protection scope and the disclosed scope of the present application.

Though the preferred embodiments of the present application have been described above in detail, the present application is not limited to details of the above-described embodiments, and various simple modifications can be made to the technical solutions of the present application without departing from the scope of the present application. These simple modifications are all within the protection scope of the present application.

In addition, it is to be noted that if not in collision, the specific technical features described in the above specific embodiments may be combined in any suitable manner. In order to avoid unnecessary repetition, the present application does not further specify any of various possible combination manners.

1. A method for preventing, delaying or treating neurodegenerative disease, comprising administering to a subject in need thereof effective amount of Prussian blue nanoparticles.

2. The method according to claim 1, wherein the neurodegenerative disease comprises Alzheimer's disease, Parkinson's disease, Amyotrophic lateral sclerosis, Huntington's disease, spinocerebellar ataxia, spinal muscular atrophy, multiple sclerosis or epilepsy.

3. The method according to claim 1, wherein the Prussian blue nanoparticles are Prussian blue nanoparticles which are functionally modified or which are non-functionally modified.

4. The method according to claim 3, wherein the Prussian blue nanoparticles are Prussian blue nanoparticles which are modified with a functional molecule which crosses the blood-brain barrier and/or a molecule which specifically targets AP deposition.

5. The method according to claim 4, wherein the functional molecule which crosses the blood-brain barrier comprises any one or any combination of at least two of transferrin, lactoferrin, apolipoprotein E, Angiopep-2, RVG29 or a TAT peptide.

6. The method according to claim 4, wherein the molecule which specifically targets AP deposition comprises any one or any combination of at least two of Congo red, thioflavin S or an anti-Aβ antibody.

7. The method according to claim 1, wherein the Prussian blue nanoparticles have a particle size of 80-200 nm.

8. The method according to claim 1, wherein the Prussian blue nanoparticles are loaded on a pharmaceutical carrier.

9. The method according to claim 8, wherein the Prussian blue nanoparticles are comprised in a medicament composition.

10. The method according to claim 1, wherein the medicament is in a dosage form comprising a tablet, a powder, a suspension, a granule, a capsule, an injection, a spray, a solution, an enema, an emulsion, a film, a suppository, a patch, a nasal drop or a pill.

11. The method according to claim 1, wherein the medicament is administrated by a route comprising intravenous injection, intraperitoneal injection, intramuscular injection, subcutaneous injection, oral administration, sublingual administration, nasal administration or transdermal administration.

12. A method for inhibiting the expression of an oxidative stress marker, comprising administering to a subject in need thereof effective amount of Prussian blue nanoparticles, wherein the oxidative stress marker comprises 4-hydroxynonenal, malondialdehyde or 8-hydroxyguanosine.

13. A medicament for the prevention, delay or treatment of neurodegenerative disease, comprising Prussian blue nanoparticles.

14. The medicament according to claim 13, further comprising a pharmaceutically acceptable adjuvant, wherein the pharmaceutically acceptable adjuvant comprises any one or any combination of at least two of a diluent, an excipient, a filler, a binder, a wetting agent, a disintegrant, an emulsifier, a cosolvent, a solubilizer, an osmotic pressure regulator, a surfactant, a pH regulator, an antioxidant, a bacteriostatic agent, or a buffer.
